(19) United States
(12) Patent Application Publication
(10) Pub. No.: US 2021/0029997 A1
(43) Pub. Date: Feb. 4, 2021

(54) ORGANIC ANTIMICROBIAL AGENT

(71) Applicants: Robert Watson, Thomastown (AU); Kevin Joyce, Thomastown (AU)

(72) Inventors: Robert Watson, Thomastown (AU); Kevin Joyce, Thomastown (AU)

(73) Assignee: Environmental Services Pty. Ltd., Thomastown (AU)

(21) Appl. No.: 16/942,600

(22) Filed: Jul. 29, 2020

(30) Foreign Application Priority Data
Aug. 2, 2019 (AU) 2019902768

(51) Int. Cl. A01N 43/08; A01N 31/02; A01N 37/02; A01N 25/30; A01N 25/06; A01N 65/00

(52) U.S. Cl. CPC A01N 43/08; A01N 31/02; A01N 65/00; A01N 25/30; A01N 25/06; A01N 37/02

(57) ABSTRACT

An organic antimicrobial agent for use as a disinfectant includes a mixture by percentage volume of:
glycerine 10-40%;
ascorbic acid 1-25%;
ethanol 1-25%;
lactic acid 1-25%;
fatty acid 1-25%; and,
surfactant 1-25%.

The organic antimicrobial agent, preferably, further includes by percentage volume:
bioflavonoids 10-40%;
saponin 1-25%; and,
ascorbic acid 1-25%.

### BACKGROUND OF THE INVENTION

### Field of the Invention

The present invention relates, generally, to an antimicrobial agent, and in particular, to an antimicrobial agent made from organic ingredients that has an adverse effect on microorganisms including bacteria, fungi and viruses.

### Description of the Prior Art

Historically, many antimicrobial agents have been found to have undesirable side-effects. As a result, there has been pressure, mainly from consumers, for a move towards safer and more “natural based” alternatives.

This change in behavior can be largely attributed to a growing global trend towards a reduction in use of chemical antimicrobial agents, such as disinfectants and sanitizers.

It is known to provide anti-bacterial agents, produced from a mixture of bioflavonoids or products made from different citrus fruit or vegetable extracts. These types of products can be generally referred to as bioflavonoids. However, such products have typically been proven to have limited antimicrobial actions.

Thus, there is a need to provide a new antimicrobial agent which has superior antimicrobial action when compared to existing products made from organic ingredients and which has not been achieved by other citrus agent formulations and which can also preferably have anti-mildew and anti-viral properties.

The above references to, and descriptions of, prior proposals or products are not intended to be, and are not to be construed as, statements or admissions of common general knowledge in the art. In particular, the above prior art discussion does not relate to what is commonly or well known by the person skilled in the art, but assists in the understanding of the inventive step of the present invention of which the identification of pertinent prior art proposals is but one part.

### SUMMARY OF THE INVENTION

Accordingly, in one preferred embodiment of the present invention, there is provided an organic antimicrobial agent for use as a disinfectant comprising a mixture by percentage volume of:
Glycerine 10-40%;
Ascorbic Acid 1-25%;
Ethanol 1-25%;
Lactic Acid 1-25%;
Fatty Acid 1-25%; and,
Surfactant 1-25%.

In another preferred embodiment of the invention, the organic antimicrobial agent further comprises
Bioflavonoids 10-40%;
Saponin 1-25%; and,
Ascorbic Acid 1-25%.

The organic antimicrobial agent may be diluted in water to form a mixture for use. In one form, the dilution rate may be 60 ml/liter of water. In another form, the dilation rate may be between 0.5 and 1.5 ml per liter of water.

In one preferred embodiment, the mixture may be applied to a surface to be treated in the form of a spray or fog.

In another preferred embodiment, the item to be treated may be dipped or soaked in the mixture.

Other objects and features of the present invention will become apparent when considered in combination with the following detailed description of the invention, which provides certain preferred embodiments and examples of the present invention. It should, however, be noted that the accompanying detailed description is intended to discuss and explain only certain embodiments of the claimed invention and is not intended as a means for defining the limits and scope of the invention.

### DETAILED DESCRIPTION OF THE INVENTION

In accordance with a preferred embodiment, the present invention provides an antimicrobial agent being a synergistic mixture of bioflavonoids together with certain organic acids and carriers. The mixture may also include alcohol, surfactant, glycerine and water. Such a mixture composition has been found to act in a synergistic manner to give a better result as an antimicrobial agent than the use of either of the compounds alone. The mixture of the present invention may also be combined with detergents and cleaners to further enhance its antimicrobial properties. In this regard, it has been found that by diluting the mixture of the present invention with water to as little as 1 ml per liter, a high antimicrobial kill rate can be achieved.

In accordance with a first preferred embodiment of the invention, the mixture comprises the following components by percentage volume:
Glycerine 10-40%;
Ascorbic Acid 1-25%;
Ethanol 1-25%;
Lactic Acid 1-25%;
Fatty Acid 1-25%; and
Surfactant 1-25%.
   In use, this mixture is diluted with water and has been found that when used on high bacterial loads, such as those typically found in hospitals, the dilution rate can be very high (up to 60 ml/liter.)

The mixture can also be used in food safety applications, where a dilation rate of between 0.5 and 1.5 ml per liter of the mixture added to water provides a satisfactory dilution.

The mixture referred to above functions by way of a process of cell wall intrusion, complexation of key cations and through the disruption of protein functioning. Through these actions the mixture of the present invention has been proven to adversely affect micro-cellular organisms, such as bacteria, and non-cellular organisms, such as viruses.

The mixture is a botanic and organic acid blend disinfectant concentrate and can be used as a disinfectant for hard surfaces with a specific application to the food and agriculture sector.

Various laboratory trials of the mixture have been undertaken against varying levels of bacteria, fungi and viruses. Bacteria testing has been conducted against a variety of bacteria, including E. coli, Listeria, MRSA, Pseudomonas and Staphylococcus. TABLE 1 depicts the results of Bacteria testing of this mixture:

TABLE 1

| | | Count | Growth in Recovery Broths | | |
|---|---|---|---|---|---|
| Test | Concentration | (Orgs/mL) | Challenge 1 | Challenge 2 | Results |

A. Escherichia coli NCTC 8196

| | | | | | |
|---|---|---|---|---|---|
| 1 | 6% | 6.2 × 10⁸ | − − − − − | − − − − − | Pass |
| 2 | 6% | 4.1 × 10⁸ | − − − − − | − − − − − | Pass |
| 3 | 6% | 8.4 × 10⁸ | − − − − − | − − − − − | Pass |

B. Proteus vulgaris NCTC 4635

| | | | | | |
|---|---|---|---|---|---|
| 1 | 6% | 5.2 × 10⁸ | − − − − − | − − − − − | Pass |
| 2 | 6% | 2.9 × 10⁸ | − − − − − | − − − − − | Pass |
| 3 | 6% | 8.4 × 10⁸ | − − − − − | − − − − − | Pass |

C. Pseudomonas aeruginosa NCTC 6749

| | | | | | |
|---|---|---|---|---|---|
| 1 | 6% | 4.5 × 10⁸ | − − − − − | − − − − − | Pass |
| 2 | 6% | 3.1 × 10⁸ | − − − − − | − − − − − | Pass |
| 3 | 6% | 5.1 × 10⁸ | − − − − − | − − − − − | Pass |

D. Staphylococcus aureus NCTC 4163

| | | | | | |
|---|---|---|---|---|---|
| 1 | 6% | 5.1 × 10⁸ | − − − − − | − − − − − | Pass |
| 2 | 6% | 4.9 × 10⁸ | − − − − − | − − − − − | Pass |
| 3 | 6% | 1.3 × 10⁹ | − − − − − | + − − − − | Pass |

Notes:
‘−’ indicates no growth in recovery broths
‘+’ indicates growth in recovery broths
The sample tested was found to pass the test under the above test conditions. All controls conformed to the requirements of the test procedure.

The mixture tested above was diluted in water to 6% and was evaluated in accordance with the Australia Therapeutic Goods Administration (TGA) Test for Hospital Grade Disinfectants. As is noted, the mixture was found to pass the test proving its effectiveness against such bacteria.

Fungi testing of the mixture has also been successfully conducted against a variety of different fungi, including Aspergillus Niger.

The mixture has also undertaken virus tests against a variety of different viruses, including H1N1 and Corona. Testing was conducted at varying concentrations of the product, to determine optimum effectiveness rates.

TABLE 2 depicts the results of testing of the mixture of the present invention against the Murine hepatitis virus VR-261 which is a member of the Coronavirus family and has been used as a surrogate for the SARS coronavirus in testing regimes. This same testing scenario exists for the now named Severe Acute Respiratory Coronavirus 2 (SARS-CoV-2), which causes the COVID-19 disease:

TABLE 2
CONDITIONS

| | |
|---|---|
| Virus Strain | Murine hepatitis virus (MHV)-1 ATCC/VR-261 |
| Cell Substrate | A9 cell ATCC/CCL-1.4 |
| Test Concentration | 6% (in hard water) |
| Contact Time | 10 minutes |
| Test Temperature | Room Temperature |
| Test Condition | Dirty 3% FBS (Fetal Bovine Serum) with 3% |
| | erythrocytes (EN14476) |
| Neutralizer | 2% FBS in MM |

TABLE 1
RESULTS: MHV-1 virus test/control results for 10 minutes contact

| | Number of | | | | |
|---|---|---|---|---|---|
| Virus | Inoculated | Virus | | | Test |
| Dilutions | Wells | Control | Cytotoxicity | Neutralisation | Sample |
| 10⁻¹ | 4 | 4*/4 | C | C | C |
| 10⁻² | 4 | 4*/4 | 0*/4 | 4*/4 | 3*/4 |
| 10⁻³ | 4 | 4*/4 | 0*/4 | 4*/4 | 2*/4 |
| 10⁻⁴ | 4 | 4*/4 | N/A | N/A | 0*/4 |
| 10⁻⁵ | 4 | 3*/4 | N/A | N/A | 0*/4 |
| 10⁻⁶ | 4 | 3*/4 | N/A | N/A | 0*/4 |
| 10⁻⁷ | 4 | 1*/4 | N/A | N/A | N/A |
| 10⁻⁸ | 4 | 0*/4 | N/A | N/A | N/A |
| Log₁₀ | — | 6.33 | 1.50 | 1.50 | 2.77 |

| | |
|---|---|
| Log₁₀ Reduction of Virus after Treatment | 3.56 |

Note:
Presence of virus in each response is recorded as “+”
Absence of virus in each response is recorded as “0”
Cytotoxic response is recorded as “C”
Calculated virus titre = 10^{6.33} TCID_{500.1 ml} (6.33 log₁₀)
Cell control - 4 wells with health cell monolayer
*The Reed & Muench LD50 Method was used for determining the virus titer endpoint.

In the test, as depicted in TABLE 2, the mixture was diluted to 6% in water and based on the cytotoxicity and neutralization test results, the mixture was found to show virucidal efficacy against MHV-1 by achieving 3.56 log reduction in virus concentration after 10 minutes exposure at room temperature. This equates to the mixture being 99.95% effective.

In order to obtain residual antimicrobial results, the mixture of the present invention can be sprayed or fogged in a light mist and left to dry on the surface to be treated. This can be done throughout small and large areas, indoors and outdoors, with no toxic, corrosive or environmental concerns. It may also be used to dip or soak for materials or objects that require reduced microbe load. The mixture is organically certified in Australia and New Zealand and all ingredients are listed as GRAS in accordance with § 201 and § 409 of the Federal Food, Drug and Cosmetic Act (USA.)

Further to this, as residues of the mixture components are not toxic; are applied in only small quantities; and are natural products, the mixture is acceptable for use in food and organic farming inputs. In any case, because of the low concentrations, the amount of residues is extremely small.

An alternative version of the mixture of the present invention is depicted below: This preferred embodiment of the mixture comprises the same elements in the same percentages as that described above, with the inclusion of some additional components, namely. bioflavonoids, saponin and ascorbic acid. The mixture is configured as follows:
Glycerine 10-40%;
Ascorbic Acid 1-25%;
Ethanol 1-25%;
Lactic Acid 1-25%;
Fatty Acid 1-25%;
Surfactant 1-25%;
Bioflavonoids 10-40%;
Saponin 1-25%; and,
Ascorbic Acid 1-25%.
   The additional components are also natural products and maintain many the benefits of the previously defined mixture. The mixture is completely natural and is a botanic and organic acid blend disinfectant concentrate and can be used as a disinfectant for hard surfaces with a specific application to the food and agriculture sector.

This mixture has also undertaken various laboratory trials against varying levels of bacteria, fungi and viruses. A list of the pathogens that this mixture has been tested against is shown in TABLE 3, below:

TABLE 3

| | TEST | Time/ | Log |
|---|---|---|---|
| PATHOGEN | Performed | Dilution | Reduction |
| Escherichia | BS EN | 5 min/@ | >5 |
| coli | 1276:2009 | .5% | |
| Salmonella | BS EN | 5 min/@ | >5 |
| choleraesuis | 1276:2009 | .5% | |
| Pseudomonas | BS 3286 | 10 min/@ | >2 |
| aeruginosa | | .05% | |
| Staphylococcus | BS EN | 5 min/@ | >2.5 |
| Aureus | 1276:2009 | .5% | |
| Staphylococcus | BS EN | 10 min/@ | >4 |
| Aureus | 1276:2009 | .5% | |
| MRSA | BSEN | 5 mins/@ | >5 |
| | 1276:1997 | 4% | |
| Listeria | BS EN | 5 mins/@ | >5 |
| monocytogenes | 1276:2009 | 0.5% | |
| Aspergillus | BS EN | 10 mins/@ | >1 |
| Niger/brasiliensis | 1650:2008 | 2% | |
| H1N1 virus | ASTM-E1053- | 10 mins/@ | Log 4 |
| | 97 | 5% | |

| | TEST | |
|---|---|---|
| PATHOGEN | Performed | No Growth Dilution |
| Legionella pneumophila | MIC | .156% |
| Brettanomyces | MIC | .0625% |
| Aspergillus | MIC | .078% |
| Niger/brasiliensis | | |
| Listeria monocytogenes | MIC | .125% |
| Pr. Vulgaris | MIC | 1.25% |

| | TEST | | |
|---|---|---|---|
| PATHOGEN | Performed | Dilution | Result |
| Staphylococcus | TGA Commercial | 2% | Pass |
| Aureus | grade | | |
| Escherichia | TGA Commercial | 2% | Pass |
| coli | grade | | |
| Escherichia | TGA Hospital | 5% | Pass |
| coli | grade | | |
| Salmonella | TGA Hospital | 5% | Pass |
| choleraesuis | grade | | |
| Pseudomonas | TGA Hospital | 5% | Pass |
| aeruginosa | grade | | |
| Pr. Vulgaris | TGA Hospital | 5% | Pass |
| | grade | | |

Table 4 depicts the results of testing of the mixture of the alternative preferred embodiment of the present invention against the Murine hepatitis virus VR-261 which is a member of the Coronavirus family and has been used as a surrogate for the SARS coronavirus in testing regimes. This same testing scenario exists for the now named Severe Acute Respiratory Coronavirus 2 (SARS-CoV-2) which causes the COVID-19 disease:

TABLE 4
CONDITIONS

| | |
|---|---|
| Virus Strain | Murine hepatitis virus (MHV)-1 ATCC/VR-261 |
| Cell Substrate | A9 cell ATCC/CCL-1.4 |
| Test Concentration | 6% (in hard water) |
| Contact Time | 10 minutes |
| Test Temperature | Room Temperature |
| Test Condition | Dirty 3% FBS (Fetal Bovine Serum) with 3% |
| | erythrocytes (EN14476) |
| Neutralizer | 2% FBS in MM |

TABLE 1
RESULTS: MHV-1 virus test/control results for 10 minutes contact

| | Number of | | | | |
|---|---|---|---|---|---|
| Virus | Inoculated | Virus | | | Test |
| Dilutions | Wells | Control | Cytotoxicity | Neutralisation | Sample |
| 10⁻¹ | 4 | 4*/4 | C | C | C |
| 10⁻² | 4 | 4*/4 | 0*/4 | 4*/4 | 2*/4 |
| 10⁻³ | 4 | 4*/4 | 0*/4 | 4*/4 | 1*/4 |
| 10⁻⁴ | 4 | 4*/4 | N/A | N/A | 0*/4 |
| 10⁻⁵ | 4 | 3*/4 | N/A | N/A | 0*/4 |
| 10⁻⁶ | 4 | 3*/4 | N/A | N/A | 0*/4 |
| 10⁻⁷ | 4 | 1*/4 | N/A | N/A | N/A |
| 10⁻⁸ | 4 | 0*/4 | N/A | N/A | N/A |
| Log₁₀ | — | 6.33 | 1.50 | 1.50 | 2.23 |

| | |
|---|---|
| Log₁₀ Reduction of Virus after Treatment | 4.10 |

Note:
Presence of virus in each response is recorded as “+”
Absence of virus in each response is recorded as “0”
Cytotoxic response is recorded as “C”
Calculated virus titre = 10^{6.33} TCID_{500.1 ml} (6.33 log₁₀)
Cell control - 4 wells with health cell monolayer
*The Reed & Muench LD50 Method was used for determining the virus titre endpoint.

In the test as depicted in TABLE 4, the mixture was diluted to 6% in water and based on the cytotoxicity and neutralization test results, the mixture was found to show virucidal efficacy against MHV-1 by achieving 4.10 log reduction in virus concentration after 10 minutes exposure at room temperature. This equates to the mixture being 99.99% effective.

As with the previously described mixture, this alternative mixture can be sprayed or fogged in a light mist and left to dry on the surface to be treated. This can be done throughout small and large areas, indoors and outdoors, with no toxic, corrosive or environmental concerns. It may also be used to dip or soak for materials or objects that require reduced microbe load. The mixture is organically certified in Australia and New Zealand and all ingredients are listed as GRAS in accordance with § 201 and § 409 of the Federal Food, Drug and Cosmetic Act (USA).

Further to this, as residues of the mixture components are not toxic; are applied in only small quantities; and are natural products, the mixture is acceptable for use in food and organic farming inputs. In any case, because of the low concentrations, the amount of residues is extremely small.

While in the specification we have described particular certain applications and percentages of components and quantities applied, it is to be understood that these are exemplary of the invention and not restrictive.

It can be seen from these results that the product is as effective as any chemical agent but has the advantages over such agent as set out above.

Variations and modifications may be made in the synergistic mixtures of the invention without departing from the spirit and scope thereof.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

While only several embodiments of the present invention have been shown and described, it will be obvious to those skilled in the art that many modifications may be made to the present invention without departing from the spirit and scope thereof.

What is claimed is:

1. An organic antimicrobial agent for use as a disinfectant, comprising a mixture by percentage volume of:
glycerine 10-40%;
ascorbic acid 1-25%;
ethanol 1-25%;
lactic acid 1-25%;
fatty acid 1-25%; and,
surfactant 1-25%.

2. The organic antimicrobial agent for use as a disinfectant according to claim 1, further comprising by percentage volume:
bioflavonoids 10-40%;
saponin 1-25%; and,
ascorbic acid 1-25%.

3. The organic antimicrobial agent for use as a disinfectant according to claim 1, wherein the organic antimicrobial agent is diluted in water to form a mixture for use.

4. The organic antimicrobial agent for use as a disinfectant according to claim 3, wherein the organic antimicrobial agent is diluted at a rate of 60 ml/liter of water.

5. The organic antimicrobial agent for use as a disinfectant according to claim 3, wherein the organic antimicrobial agent is diluted at a rate of between 0.5 and 1.5 ml/liter of water.

6. A method for using an organic antimicrobial agent as a disinfectant, said organic antimicrobial agent comprising a mixture by percentage volume of:
glycerine 10-40%;
ascorbic acid 1-25%;
ethanol 1-25%;
lactic acid 1-25%;
fatty acid 1-25%; and,
surfactant 1-25%,
said method for using said organic antimicrobial agent as a disinfectant comprising the steps of:
diluting said organic antimicrobial agent to form a mixture; and,
applying said mixture comprising said organic antimicrobial agent to a surface of an item to be disinfected.

7. The method for using the organic antimicrobial agent for use as a disinfectant according to claim 6, wherein said organic antimicrobial agent further comprises:
bioflavonoids 10-40%;
saponin 1-25%; and,
ascorbic acid 1-25%.

8. The method for using the organic antimicrobial agent for use as a disinfectant according to claim 6, wherein said step of diluting said organic antimicrobial agent to form a mixture is carried out by diluting said organic antimicrobial agent in water.

9. The method for using the organic antimicrobial agent for use as a disinfectant according to claim 8, wherein said organic antimicrobial agent is diluted at a rate of 60 ml/liter of water.

10. The method for using the organic antimicrobial agent for use as a disinfectant according to claim 8, wherein said organic antimicrobial agent is diluted at a rate of between 0.5 and 1.5 ml/liter of water.

11. The method for using the organic antimicrobial agent for use as a disinfectant according to claim 6, wherein said step of applying said mixture comprising said organic antimicrobial agent to a surface of an item to be disinfected is carried out applying said mixture to the surface of the item in the form of a spray or fog.

12. The method for using the organic antimicrobial agent for use as a disinfectant according to claim 6, wherein said step of applying said mixture comprising said organic antimicrobial agent to a surface of an item to be disinfected is carried out applying said mixture to the surface of the item by dipping or soaking the item in the mixture.

13. The method for using the organic antimicrobial agent for use as a disinfectant according to claim 6, wherein said step of applying said mixture comprising said organic antimicrobial agent to a surface of an item to be disinfected is carried out applying said mixture to the surface of the item by brushing the mixture onto the surface of the item.
